(19) United States
(12) Patent Application Publication
(10) Pub. No.: US 2026/0105996 A1
(43) Pub. Date: Apr. 16, 2026

(54) SYSTEMS AND METHODS FOR PREDICTING TABLETABILITY OF MIXTURES

(71) Applicant: Regents of the University of Minnesota, Minneapolis, MN (US)

(72) Inventors: Changquan Calvin Sun, West Lafayette, IN (US); Gerrit Vreeman, Excelsior, MN (US)

(73) Assignee: Regents of the University of Minnesota, Minneapolis, MN (US)

(21) Appl. No.: 19/352,915

(22) Filed: Oct. 8, 2025

Related U.S. Application Data
(60) Provisional application No. 63/705,672, filed on Oct. 10, 2024.

(51) Int. Cl. G16C 20/30; G16C 20/70; G16C 60/00

(52) U.S. Cl. CPC G16C 20/30; G16C 20/70; G16C 60/00

(57) ABSTRACT

Methods include receiving from a formulator a proposed formulation comprising a plurality of components. Tabletability parameters for each component of the plurality of components are retrieved, for example from a material database. A predicted tabletability profile is generated for the proposed formulation based upon the tabletability parameters of each component of the plurality of components and a composition of the proposed formulation. The predicted tabletability profile is conveyed to the formulator. The systems and methods of the present disclosure are useful with binary and more complex (i.e., three or more components) mixtures.

### CROSS REFERENCE TO RELATED APPLICATIONS

This application claims priority to U.S. Provisional Patent Application No. 63/705,672 filed Oct. 10, 2024, entitled “SYSTEMS AND METHODS FOR PREDICTING TABLETABILITY OF MIXTURES” and entirety of which is incorporated herein by reference.

### GOVERNMENT SUPPORT CLAUSE

This invention was made with government support under IIP2137264 awarded by the National Science Foundation. The government has certain rights in the invention.

### BACKGROUND

The present disclosure relates to tablet formulation development. More particularly, it relates to systems and methods for predicting the tabletability behaviors of a mixture of two or more components, such as a mixture or compound under consideration for development as a useful pharmaceutical tablet formulation.

Oral solid dosage forms, such as tablets, capsules, powders, etc., are administered to achieve local or systemic therapeutic effects in the body after absorption. Tablets have become a popular dosage form for delivering small-molecule drugs owing to various advantages, such as economy, excellent stability, and high patient compliance. Tablet formulation development consists of identifying suitable formulation composition and manufacturing process parameters. In a tablet, the active pharmaceutical ingredients (API) imparts therapeutic effects while tablet excipients ensure the manufacturability and functionality of the final dosage form. Excipients are commonly formulation aids, e.g., diluents, binders, disintegrants, glidants, and lubricants, used to ensure successful manufacturing of tablets. However, some excipients are used to attain specific functions, e.g., modifying drug release, enhancing solubility, masking taste, or increasing tablet residence time by mucoadhesion. Excipients selected should be chemically stable and compatible with the intended API.

The tablet manufacturing process can be complex, involving multiple unit operations, such as granulation (dry, wet, or fluid-bed), mixing, drying, milling and coating, to ensure robust production of tablets meeting pharmacopoeial quality standards, such as friability, stability, and dissolution. Formulation and process are developed in laboratory through conducting a series of experiments and then scaled up for commercialization upon gaining FDA approval based on therapeutic efficacy and safety of the drug product. In summary, the current approach to tablet formulation in the pharmaceutical industry largely follows empirical, design-of-experiments (DOE) based approaches for optimization. This approach is both time and materials intensive, making it unfit for tablet formulation development early in drug product development due to the limited availability of active pharmaceutical ingredients (API).

Tabletability is the relationship between tablet tensile strength and the compaction pressure used to produce the tablet. A sufficiently high tensile strength is required to withstand the stresses that a tablet encounters throughout its lifetime, but this must be balanced with competing properties imperative to a formulated product's success, like disintegration. Ensuring that a formulation has appropriate tabletability is a key part of formulation development. Current methods for formulation tabletability optimization suffer from the material-intensive, empirical, DOE based approaches

### SUMMARY

The inventors of the present disclosure have recognized a need to address one or more of the above-mentioned problems.

Some aspects of the present disclosure relate to methods that include receiving from a formulator a proposed formulation comprising a plurality of components. Tabletability parameters for each component of the plurality of components are retrieved, for example from a material database. A predicted tabletability profile is generated for the proposed formulation based upon the tabletability parameters of each component of the plurality of components and a composition of the proposed formulation. The predicted tabletability profile is conveyed to the formulator. In some examples, the tabletability parameters for each of the components include a maximum strength attainable by the material upon complete densification (σₘₐₓ); a sigmoidal profile shift influencing the onset of appreciable mechanical strength gain (α); and a material plasticity (β). With these and related embodiments, the predicted tabletability profile is derived from predicted tabletability parameters of the proposed formulation that are based upon the corresponding tabletability parameter of each of the constituting components and a composition of the components in the proposed formulation. In some examples, the tabletability parameters are determined from the equation:

$$σ = {σ}_{m ax} {e}^{α {W}^{( - {e}^{\frac{- P}{β} - 1} )}}$$

and the tabletability parameters of the proposed formulation are determined by:

$${X}_{mix} = {\prod }_{i = 1}^{n} {X}_{i}^{{C}_{i}}$$

where Xₘᵢₓ is the tabletability parameters of a given mixture, Xᵢ is the parameter of an individual component, and Cᵢ is composition of individual components (e.g., volume fraction, weight fraction, etc.). The systems and methods of the present disclosure are useful with a wide variety of proposed mixtures having a plurality of components (e.g., two, three, four, or more components).

### BRIEF DESCRIPTION OF THE DRAWINGS

FIG. 1 is a block diagram of a system for screening or predicting tabletability of a potential formulation in accordance with principles of the present disclosure;

FIG. 2 is a plot illustrating a tabletability profile of microcrystalline cellulose Avicel PH102;

FIG. 3A is a plot of a simulated effect of a tabletability parameter (σₘₐₓ) in accordance with principles of the present disclosure;

FIG. 3B is a plot of a simulated effect of another tabletability parameter (α) in accordance with principles of the present disclosure;

FIG. 3C is a plot of a simulated effect of another tabletability parameter (β) in accordance with principles of the present disclosure;

FIG. 4 is a flow diagram of a method in accordance with principles of the present disclosure;

FIG. 5 is a flow diagram of a method in accordance with principles of the present disclosure; and

FIGS. 6-27D illustrate results described in the Examples section.

### DETAILED DESCRIPTION

Some aspects of the present disclosure are directed to systems and methods for automatically screening or predicting tabletability of potential formulations. When utilized as part of a pharmaceutical tablet formulation development process, the tabletability of a large number of different potential formulations can be quickly obtained, with the potential formulation(s) exhibiting desired predicted tabletability properties then being selected for further investigation (e.g., an identified lead formulation can be verified and optimized by running a few experiments).

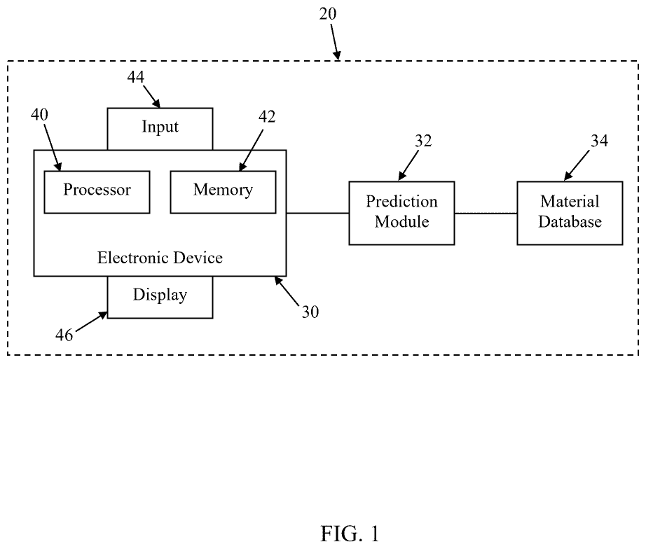

One example of an evaluation system 20 in accordance with principles of the present disclosure, and useful for performing methods of the present disclosure, is shown in block form in FIG. 1. The system 20 includes an electronic device 30, a prediction module 32 and a material database 34. Details on the various components are provided below. In general terms, parameters of a proposed formulation including two or more materials are inputted by a user at the electronic device 30. The prediction module 32 is configured or programmed to receive the inputted parameters, retrieve properties for each of the two or more materials from the material database 34, apply the modeling or equations described below to the proposed formulation, and generate predicted tabletability properties of the proposed formulation. The so-generated tabletability properties are conveyed to the user at the electronic device 30. While FIG. 1 generally reflects the prediction module 32 and the material database 34 as residing apart from the electronic device 30, in other embodiments, one or both of the prediction module 32 and the material database 34 can be provided with (e.g., programmed to) the electronic device 30.

The electronic device 30 includes a processor 40 coupled to one or more memories 42. The processor 40 can be a microprocessor, an embedded microprocessor, an embedded controller, a digital signal processor (DSP), etc. The processor 40 is configured to execute program code stored in the memory 42 (e.g., registers, cache, random-access memory, read-only memory, EEPROM, flash memory, USB drives, or the like or combinations thereof). The memory 42 can include volatile or non-volatile memory or other non-transitory computer-readable medium storing one or more instruction sets. The memory 42 can include a non-volatile computer readable medium containing computer code that, when executed by the controller 40, creates an executing environment for implementing various operations, including one or more of the computer-implemented methods of the present disclosure (e.g., steps set forth in the various flow charts and other algorithmic descriptions as set forth herein), as well as other operations. The program code, when executed by the processor 40, causes the processor 40 to implement the various functions described herein. The processor 40 can reside in any suitable computing equipment, such as a personal computer, laptop, mobile electronic device (e.g., smart phone), server or cloud-based computational platform. The processor 40 can further cooperate with the memory 42 to store data received from or generated by the prediction module 32. Regardless, the electronic device 30 includes one or more input devices 44 appropriate for receiving proposed formulation parameters from a user (e.g., a key board, touch screen, etc.), and one or more display devices 46 appropriate for conveying predicted tabletability properties of the proposed formulation to the user (e.g., a display screen, graphical user interface, etc.).

The prediction module 32 comprises program code operable by a processor to retrieve information relevant to a proposed formulation from the material database 34 and to apply the modeling or equations described below to generate predicted tabletability properties of a proposed formulation. The computer-implemented methods of the present disclosure associated embodied by the prediction module 32 operate one or more algorithms, equations, or modelings as described below. In some examples, the prediction module 32 can be provided with the electronic device 30 (e.g., as program code stored in the memory 42 and operated by the processor 40). In other embodiments, the prediction module 32 can reside on a remote computer-type device apart from the electronic device 30 as described below.

The material database 34 includes determined or known tabletability properties for a number of different materials. In some embodiments, the tabletability properties provided with the material database 34 for each of the various materials of the database 34 can include known or obtained compaction pressure versus tensile strength data points for each material, a plot or curve of tensile strength as a function of compaction pressure for each of the various materials of the database 34 (with the plot or curve having been generated or determined from known or obtained compaction pressure versus tensile strength data points), and/or an equation representing a plot or curve of tensile strength as a function of compaction pressure for each of the various materials of the database 34. In yet other examples, the tabletability properties provided with the material database can entail known tabletability data (e.g., tensile strength versus main compaction pressure) fitted to an equation as described below.

In some examples, the prediction module 32 and the material database 34 are provided with, and operated by, the same computer-type device. For example, the prediction module 32 and the material database 34 can be provided with the electronic device 30 (e.g., stored in the memory 42 and operated by the processor 40). In other embodiments, one or both of the prediction module 32 and the material database 34 can reside on a remote computer-type device apart from the electronic device 30. For example, one or both of the prediction module 32 and the material database 34 can be programmed to, or stored by, a server computer or similar device including one or more processors and one or more memories having any of the forms described above. In some non-limiting embodiments, the prediction module 32 is provided with the electronic device 30 and the material database 34 is maintained by a remote server computer. In other non-limiting embodiments, the prediction module 32 and the material database 34 are maintained by a remote server computer. Regardless, with these and related embodiments, the electronic device 30 and the remote computer-type device include appropriate communication interface components for connecting in a communicating relationship via wired or wireless communication. This may include remote resources accessible through the Internet and/or local resources available using short range communication protocols using, e.g., physical connections (e.g., Ethernet), radio frequency communications (e.g., Wi-Fi, Bluetooth®), optical communications (e.g., fiber optics, infrared, or the like), ultrasonic communications, or any combination thereof of these or other media that might be used to carry data between the processor(s) 40 and other devices. This may also include hardware/software for a WiMax connection of a cellular network connection (using, e.g., CDMA, GSM, LTE, or any other suitable protocol or combination of protocols). This may also include hardware/software for a USB or similar connection.

The computer implemented methods of the present disclosure operate one or more algorithms or equations described below. In general terms, the models or algorithms of the present disclosure generate or provide predicted tabletability properties for a proposed formulation. As a point of reference, tabletability refers to the ability of a powder to form a tablet of specified tensile strength under the effect of applied compaction pressure. In the pharmaceutical industry, mechanical strength is commonly quantified by the maximum force required to break a tablet by diametrical compression. However, the breaking force depends on tablet size, e.g., thickness and diameter. Thus, tensile strength can be a better parameter to quantify tablet mechanical strength since it is essentially breaking force normalized by tablet size. A tabletability profile can be obtained by plotting tensile strength (σ) as a function of compaction pressure (P). Stated otherwise, tensile strength (σ) can be a key performance parameter in tablet formulation, and it can be characterized through a tabletability plot (i.e., σ versus P).

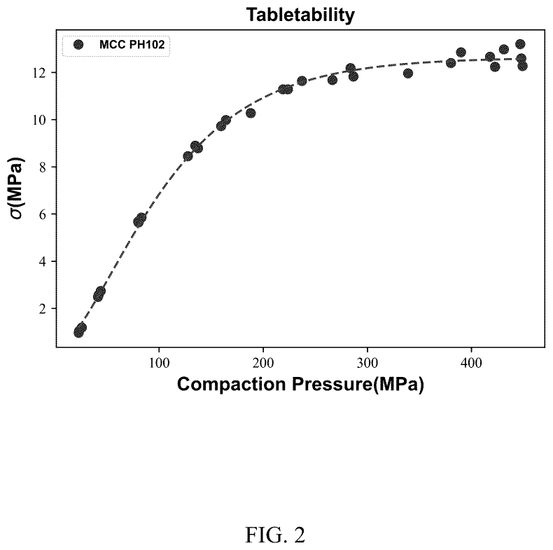

A complete tabletability profile has an asymmetric sigmoidal shape. One example is shown in FIG. 2 for the material microcrystalline cellulose Avicel PH102 (MCC PH102).

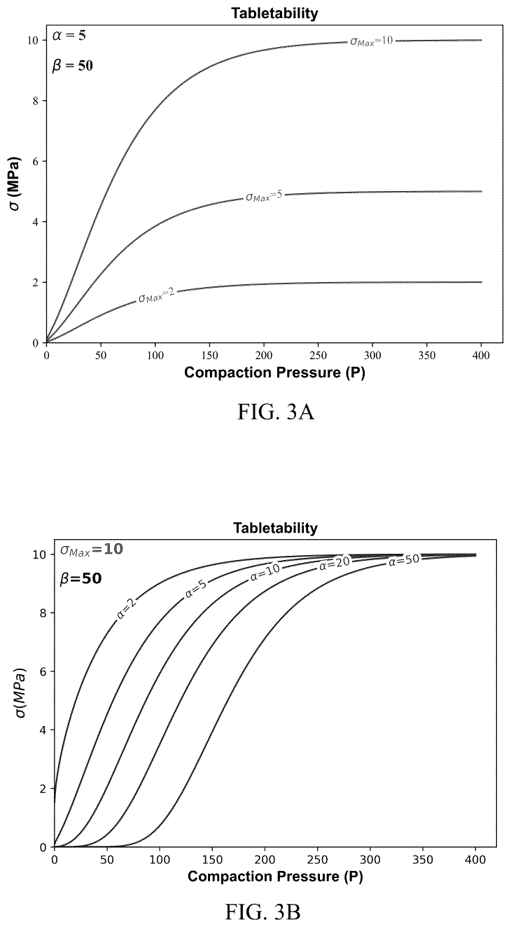

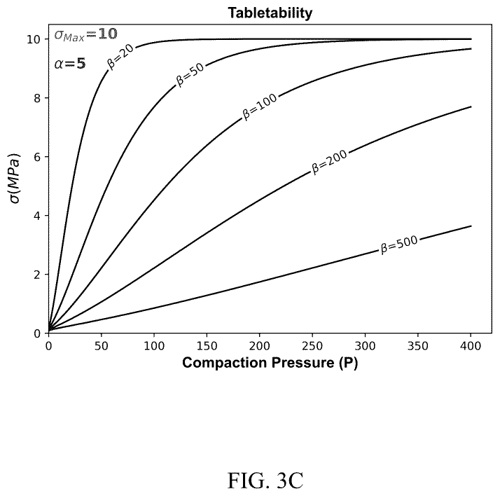

Tabletability can be qualitatively explained by the interplay between bonding area (BA) and bonding strength (BS). The BA reflects the contribution to tablet strength by the inter-particulate area formed upon the deformation of particles due to applied P. The BS reflects the contribution to tablet strength by various intermolecular forces when molecules in adjacent particles are brought sufficiently close to each other. Based on the BA-BS interplay concept, Vreeman and Sun (Vreeman G, Sun C C. A powder tabletability equation. Powder Technol. 2022; 408:117709) developed a double exponential model, by combining a compressibility equation and a compactibility equation, which contains three material-specific parameters σₘₐₓ, α, and β. The effect of each parameter on the overall tabletability profiles is simulated while keeping the other two parameters constant as reflected, for example, by FIGS. 3A-3C. Here, σₘₐₓ defines the maximum tensile strength attainable by the material upon complete densification, i.e., at zero porosity. A larger σₘₐₓ corresponds to a higher maximum tablet tensile strength that can be attained by compressing a powder (FIG. 3A). The parameter, α, defines the profiled shift influencing the onset of appreciable mechanical strength gain. That is to say, a material with a higher a requires a higher compaction pressure to form an intact tablet with measurable mechanical rigidity (FIG. 3B). The β parameter describes powder plasticity, which correlates with in-die mean yield pressure (P_{y}). With increasing compaction pressure, a tablet of a more plastic material approaches zero porosity more rapidly, leading to faster rise of tensile strength to the maximum value (FIG. 3C). As an example, the fitting of the entire tabletability profile of MCC by the Vreeman-Sun equation is shown in FIG. 2.

Compacted tablets must be sufficiently strong to withstand the stress encountered during coating, packing, shipping, and storage. Insufficient strength can result in physically defective tablets, which leads to real or perceived quality issues of the drug products. Hence, the ability to predict the tabletability of a given formulation becomes imperative to guide the efficient optimization of tablet formulation to ensure adequate mechanical strength. In fact, accurate prediction of tabletability of powder mixtures from those of pure components is an essential requirement for realizing digital design of tablets, where time- and material-intensive laborious optimization studies are eliminated. Returning to FIG. 1, the prediction module 32 is configured or programmed to predict the tabletability of a proposed mixture or formulation based upon tabletability-related properties of the mixture's constituents that are otherwise provided by the material database 34. In some non-limiting examples, the modeling or prediction algorithm(s) of the prediction model 32 directly predict tabletability without involving compressibility or compactibility. In some embodiments, the systems and methods of the present disclosure implement the Vreeman-Sun equation (Equation (1)) as the basis for predicting tabletability of mixtures from individual components.

$$σ = {σ}_{m ax} {e}^{α {W}^{( - {e}^{\frac{- P}{β} - 1} )}} \qquad EQ . ( 1 )$$

More particularly, in some embodiments the tabletability profile of an individual material or component is generated by fitting obtained or known tensile strength (σ) versus compaction pressure (P) data for the material to Equation 1 (the Vreeman-Sun equation). From this equation fitting, tabletability parameters of: maximum tensile strength (or apparent bonding strength parameter) attainable by the material upon complete densification, i.e., at zero porosity (σₘₐₓ); the profile shift influencing the onset of appreciable mechanical strength gain (or particle rearrangement parameter) (α); and a material plasticity parameter (β) are obtained. In some examples, the tabletability parameters of the present disclosure can further include effects of particle size on tabletability. For example, the parameters σₘₐₓ, α, and β can be determined for a designated particle size or range of particle sizes for the material in question.

In some examples, the tabletability parameters σₘₐₓ, α, and β for an individual material are predetermined and saved in the material database 34, optionally with a corresponding material particle size or particle size range. In other examples, the known tensile strength versus compaction pressure data for an individual material are saved in the material database 34, and the prediction module 32 is programmed or configured to determine the tabletability parameters σₘₐₓ, α, and β for the individual material by fitting the data with Equation (1) by non-linear regression. Regardless, the prediction module 32 is programmed or configured to predict the tabletability parameters of a proposed mixture or formulation (σₘₐₛₘᵢₓ, αₘᵢₓ, and βₘᵢₓ) of two or more individual materials based upon the tabletability parameters of those individual materials. In this regard, the prediction module 32 can implement or apply a mixing model or equation to determine the proposed mixture tabletability parameters. In one embodiment, the prediction module 32 utilizes Equation 2 below (sometimes referred to as the “Power” mixing rule):

$${X}_{mix} = {\prod }_{i = 1}^{n} {X}_{i}^{{C}_{i}} \qquad EQ . ( 2 )$$

where X is each of the three parameters of a given mixture, and Cᵢ is composition of individual components (e.g., volume fraction of each component, weight fraction of each component, relative amount of each individual component in the mixture, etc.). For example, the predicted apparent bonding strength, σₘₐₓₘᵢₓ, of a proposed mixture consisting of 60% component A and 40% component B could be determined from Equation 2 as:

$${σ}_{maxmix} = {σ}_{maxA}^{0.6} \times {σ}_{maxB}^{0.4}$$

In other embodiments, the prediction module 32 can be formatted to apply other mixing equations or rules to predict the proposed mixture tabletability parameter, for example one or both of Equation 3 (sometimes referred to as the “Linear” mixing rule) or Equation 4 (sometimes referred to as the “Harmonic” mixing rule) below.

$${X}_{mix} = {\sum }_{i = 1}^{n} {X}_{i} {C}_{i} \qquad EQ . ( 3 )$$

$${X}_{mix} = {( {\sum }_{i = 1}^{n} \frac{{C}_{i}}{{X}_{i}} )}^{- 1} \qquad EQ . ( 4 )$$

The prediction module 32 is further configured or programmed to generate a predicted tabletability profile or similar properties for a proposed mixture based upon the so-determined or predicted tabletability parameters of the proposed mixture or formulation (σₘₐₛₘᵢₓ, αₘᵢₓ, and βₘᵢₓ). In some examples, the tabletability profile for a proposed mixture is generated by evaluating Equation 1 (the Vreeman-Sun equation) with the predicted tabletability parameters of the proposed mixture or formulation (σₘₐₛₘᵢₓ, αₘᵢₓ, and βₘᵢₓ). The predicted tabletability profile (or other predicted tabletability-related information) generated by the prediction module 32 can be presented to a user for further evaluation.

It is recognized that some APIs are poorly compressible and do not readily form tablets (e.g., acetaminophen, alanine, etc.). Thus, it may be difficult to obtain tabletability properties of a poorly compressible API as an individual component material. With this in mind, with some embodiments of the present disclosure, binary mixtures of a poorly compressible API in question with a compressible material, such as but not limited to microcrystalline cellulose (MCC), can be prepared (with the binary mixtures having different loadings of the poorly compressible API in question) and tableted. Tabletability data for the binary mixture tablets can then be obtained and fitted to Equation 1 (the Vreeman-Sun equation) to determine the three tabletability parameters σₘₐₓ, α, and β as described above. The so-determined tabletability parameters can be plotted as a function of the poorly compressible API material loading in the mixture. Hypothetical tabletability parameters of the pure, poorly compressible API can then be determined by extrapolation (e.g., extrapolating the function to API ratio of 1 yields the hypothetical parameter for the pure, poorly compressible API). The hypothetical parameters can then be utilized as the tabletability properties for the poorly compressible API in the material database 34.

In some examples, the tabletability properties as provided with the material database 34 for a particular material can be representative of the particular material alone. In other examples, the tabletability properties provided with the material database 34 for a particular material can be or include tabletability properties for the particular material mixed with a designated lubricant at a designated level.

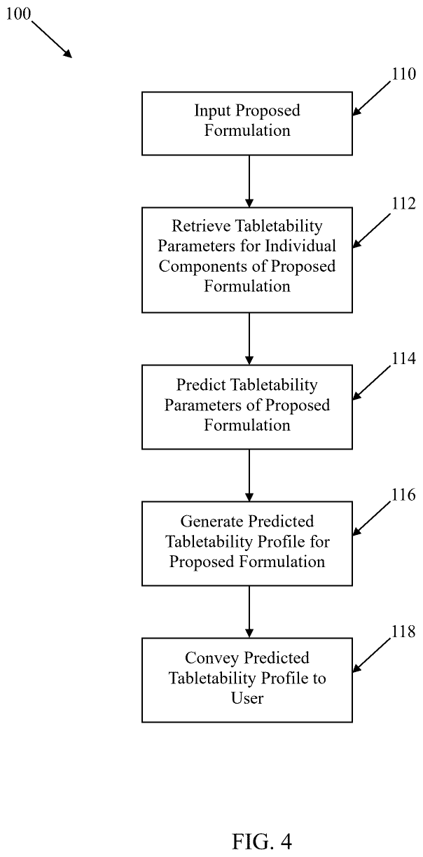

FIG. 4 illustrates example methods 100 of the present disclosure. With cross-reference between FIGS. 1 and 4, some methods of the present disclosure can include a user providing or inputting a proposed formulation or mixture via the input device 44 of the electronic device 30, including the individual component materials or constituents and relative composition (e.g., volume faction, w/w basis) at 110. The inputted proposed formulation can optionally further include an indication of particle size for one or more or all of the individual component materials. The prediction module 32 retrieves tabletability properties for each of the individual component materials or constituents of the proposed formulation from the material database 34 at 112. In this regard, the tabletability properties as stored by the material database 34 and provided to the prediction module 32 for one or more or all of the individual component materials (and/or an individual component material mixed with a designated lubricant at a designated loading level) can be the tabletability parameters σₘₐₓ, α, and β as described above. In other examples, the tabletability properties as stored by the material database 34 and provided to the prediction module 32 for one or more or all of the individual component materials can be tensile strength versus compaction pressure data for the corresponding material, and the prediction module 32 is programmed or configured to determine the tabletability parameters σₘₐₓ, α, and β for the material. Regardless, at 114, the prediction module 32 operates to predict the tabletability parameters of the proposed formulation (σₘₐₓₘᵢₓ, αₘᵢₓ, and βₘᵢₓ) based upon the tabletability parameters of the individual component materials as described above (e.g., by applying Equation (2)). At 116, the prediction module 32 operates to generate a predicted tabletability profile or similar properties for the proposed formulation based upon the predicted tabletability parameters of the proposed formulation (σₘₐₓₘᵢₓ, αₘᵢₓ, and βₘᵢₓ) as described above, for example by evaluating Equation 1 (the Vreeman-Sun equation) with the predicted tabletability parameters of the proposed formulation (σₘₐₓₘᵢₓ, αₘᵢₓ, and βₘᵢₓ). At 118, the prediction module 32 operates to convey or provide the predicted tabletability profile to the user, for example via the display device 46.

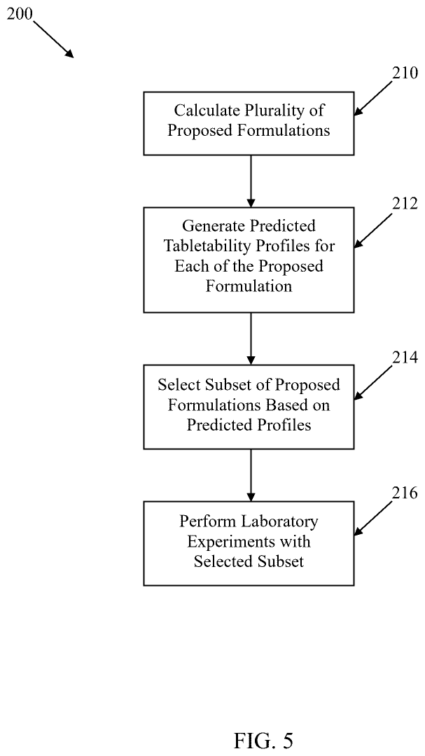

The systems and methods implicated by FIGS. 1 and 4 readily facilitate tablet formulation design. The systems and methods of the present disclosure can be highly useful to the pharmaceutical industry by significantly expediting the development of manufacturable tablet formulation while minimizing the use of API. In some embodiments, systems and methods of the present disclosure provide a materials science-based approach to tablet formulation development following quality-by-design (QbD) principals. For example, and with additional reference to FIG. 5, an example method 200 of the present disclosure can include a formulator or user considering a formulation of a proposed drug-excipient mixture in tablet form can calculate a plurality of slightly different, proposed formulations at 210. At 212 a predicted tabletability profile is generated for each of the proposed formulations, for example via the method 100 (FIG. 4) described above. At 214, the formulator evaluates each of the predicted tabletability profiles and selects a subset, optionally a single one, of the proposed formulations for further consideration based upon a best fit of the corresponding predicted tabletability profile with the formulator's desired results. Optionally, in some embodiments, the prediction module 32 can be programmed or formatted to highlight or otherwise suggest to the formulator a subset, optionally a single one, of the proposed formulations for further consideration. Regardless, at 216, the formulator performs laboratory experiments on only the selected subset of proposed formulations to confirm viability. Optionally, only one of the proposed formulations (e.g., the proposed formulation with a corresponding predicted tabletability profile closest to a desired end result) is subjected to lab experiments. Once viability is confirmed, the formulation development process continues using the confirmed proposed formulation.

### EXAMPLES

Embodiments and advantages of features of the present disclosure are further illustrated by the following non-limiting examples. The particular materials and amounts thereof recited in these examples, as well as operating conditions and details, should not be construed to unduly limit the scope of the present disclosure.

Examples were prepared and testing was performed using various obtained materials. The obtained materials included two different grades of Microcrystalline cellulose (MCC; Avicel PH102 and Avicel PH200, DuPont Nutrition Ireland, Cork, Ireland), hydroxypropyl methylcellulose (HPMC K100M DC, Ashland), hydroxypropyl methylcellulose (HPMC K100M, Colorcon, Netherlands), Kollidon VA64 (BASF, Ludwigshafen, Germany), Starch 1500 (Colorcon, Netherlands), three different grades of lactose monohydrate (LM) (Pharmatose 350M, Pharmatose 110M), lactose anhydrous (L) (Supertab 24AN, DFE Pharma, Klever Strasse, Germany), three different grades of mannitol (MN) (Pearlitol 400DC, 200SD, Roquette America Inc., Keokuk, IA), dicalcium phosphate anhydrous (DCPA; Emcompress, JRS Pharma, Patterson, NY), and theophylline anhydrate (BASF, Ludwigshafen, Germany).

Five classes of mixtures, i.e., plastic-plastic, plastic-brittle, plastic-viscoelastic, brittle-brittle, and brittle-viscoelastic, were prepared in 20% increments. Each mixture was blended using a mixer (Turbula, Glen Mills, Clifton, NJ) running at 49 revolutions per minute (rpm) for 5 minutes (min and stored in a 33% relative humidity (RH) humidity chamber for at least 24 hours before being compressed.

Tablets were compressed on a compaction simulator (Styl'One Evolution, MedelPharm, Beynost, France), simulating a Korsch XL100 compression cycle at 20 rpm, corresponding to a dwell time of 103 millisecond (ms), with a 10 megapascal (MPa) precompression step. The main compaction pressure varied between 25 and 450 MPa. Round, flat-faced (11.28 millimeter (mm) in diameter) punches were used for all mixtures except 100% MCC, HPMC, Kollidon VA64, MN 200SD, lactose 24AN, 80-20 and 60-40 mixtures of MCC, HPMC, Kollidon VA64, MN 200SD, and Lactose 24SD with Starch 1500, MCC, HPMC with LM, Mannitol, and DCPA. For these materials, 6.0 mm round, flat-faced tooling was used to lower the forces required to break the tablets compressed at high pressures. External lubrication of the die wall and punch tips using magnesium stearate spray (Styl'One MIST) was applied before each compression to lower the ejection force.

Using Equation (5) below, tablet tensile strength was calculated from tablet dimensions, measured using a digital caliper (model CD-6″ AX, Mitutoyo, Kawasaki, Kanagawa, Japan) and tablet breaking force (F), measured using a texture analyzer (TA-XT2i; Texture Technologies Corporation, Scarsdale, NY).

$$σ = \frac{2 F}{π Dt} \qquad EQ . ( 5 )$$

Where D is tablet diameter, and t is tablet thickness.

The true density of anhydrous solids was determined using a helium pycnometer (Quantachrome Instruments, Instruments, Ultrapycnometer 1000e, Boynton Beach, Florida) with an adequately weighed sample filling about 70%-75% of the volume of the sample cell). An analytical balance (Mettler Toledo, Columbus, Ohio, model AG204) was used to determine the mass. The experiment was stopped when the coefficient of variation for five consecutive measurements was below 0.005%, and the mean of the last five measurements was taken as the measured true density. For water-containing solids or hydrates, true density was obtained by fitting Equation (6) below to tablet density-pressure data.

$$P = \frac{1}{C} [ ( 1 - {ε}_{c} ) - \frac{{ρ}_{tablet}}{{ρ}_{true}} - {ε}_{c} ln ( \frac{1 - \frac{{ρ}_{tablet}}{{ρ}_{true}}}{{ε}_{c}} ) ] \qquad EQ . ( 6 )$$

An in-die Heckel analysis was performed using in-die tablet porosity, calculated from the in-die tablet thickness and weight of the ejected tablet. Mean yield pressure (P_{y}) was obtained using linear regression of the linear portion of the Heckel plot (−ln (ε) vs. P) according to Equation (7) below.

$$- ln ( ε ) = \frac{1}{{P}_{y}} P + A \qquad EQ . ( 7 )$$

The Vreeman-Sun equation (Equation (1) above) was fitted to the experimental tabletability profiles using non-linear regression to obtain three tabletability parameters, σₘₐₓ, α, and β. Non-linear regression was performed in Python (Spyder IDE 3.9) using SciPy's (v1.8.0) orthogonal distance regression using ordinary least-squares optimization. From SciPy's special function, the Lambert W function was applied, and principal branch solutions were selected as default.

Tabletability parameters of the constituent powders were used to estimate the tabletability parameters of the mixtures using the Power mixing law (Equation (2) above) without making any assumptions. The volume fraction (ϑ) of each component in the mixture was calculated using Equation (8) below, where ρᵢ is the true density of component i, and xᵢ is mass fraction of component i.

$${ϑ}_{i} = \frac{( \frac{{x}_{i}}{{ρ}_{i}} )}{{\sum }_{j = 1}^{n} ( \frac{{x}_{j}}{{ρ}_{j}} )} \qquad EQ . ( 8 )$$

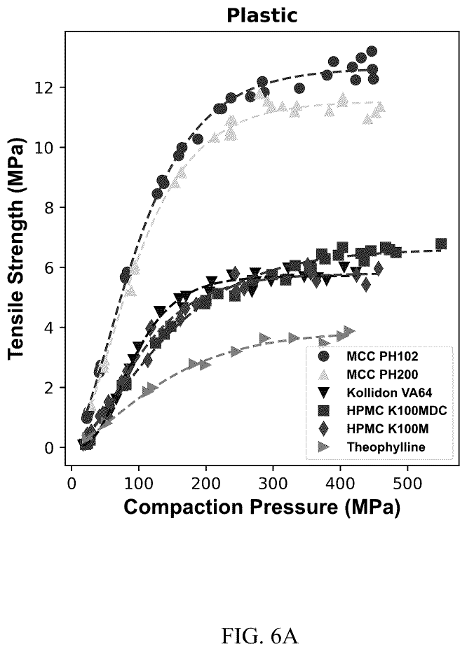

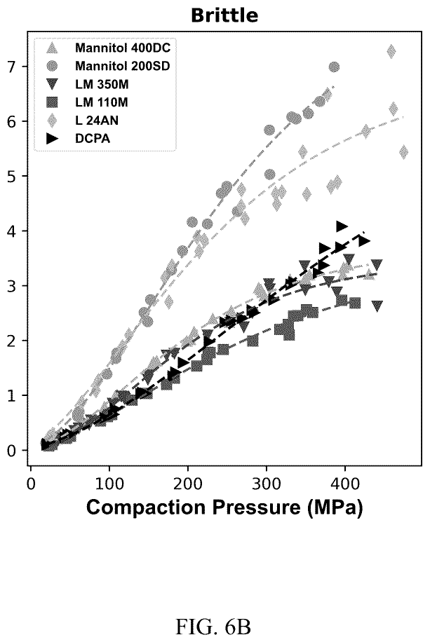

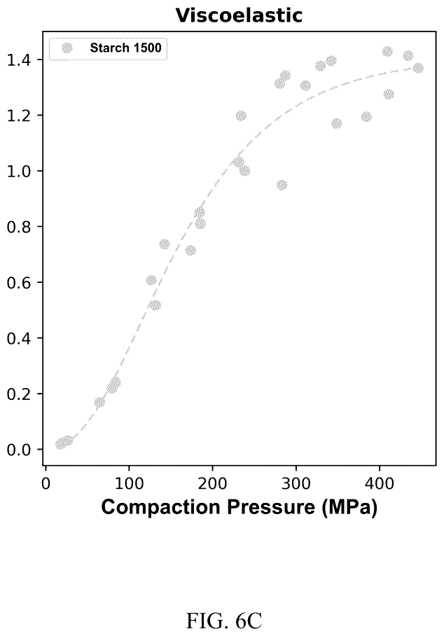

The tabletability profiles of all excipients and an API (theophylline) of the Examples section (as pure powders) were found to be well described by the Vreeman-Sun equation (Equation (1) above) and are reported in FIGS. 6A-6C. The fitting is reflected by lines in the views. To assess the performance of the predictions, the predicted tabletability profiles of mixtures using the Power mixing rule is plotted (solid lines) against the experimental tabletability data (open markers). Although the binary mixtures were prepared in 20% increments by weight, it is expressed in volume fraction when applying the mixing model as the true density can potentially vary significantly between the components (1.1-2.82 grams/cubic centimeter (g/cm³)).

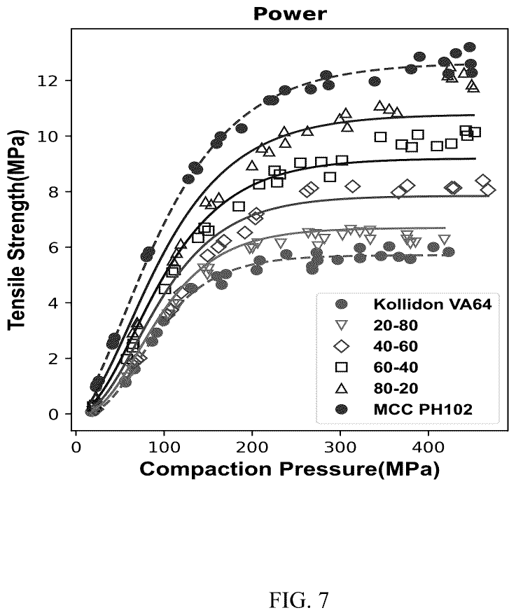

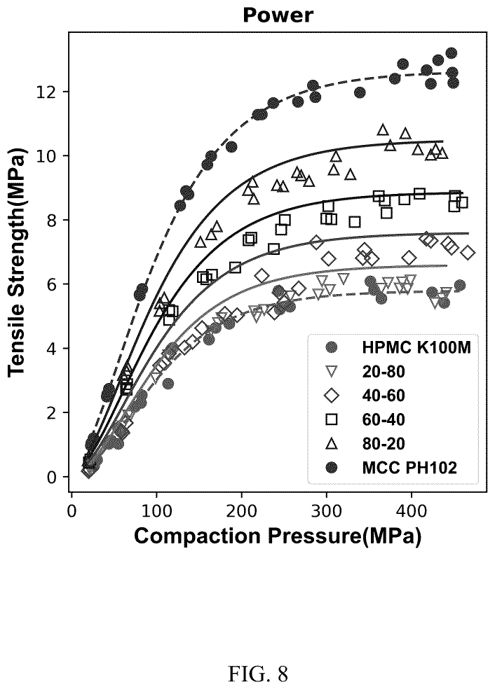

The tabletability profiles (as determined according the explanations above) of the pure components (dashed lines) and mixtures for MCC PH102-Kollidon VA64 is shown in FIG. 7. The tabletability profiles (as determined according to the explanations above) of the pure components (dashed lines) and mixtures for MCC PH102-Kollidon VA64 and MCC PH102-HPMC K100M systems are shown in FIG. 7 and FIG. 8, respectively. For the MCC PH102-Kollidon VA64 mixtures (20-80, 40-60, 60-40, 80-20), the power law mixing rule was found to predict tabletability profiles that are close to the experimental data (FIG. 7). For the MCC PH102-HPMC mixtures (20-80, 40-60, 60-40, 80-20), the power mixing rule was found to provide good predictions compared to the experimental data (FIG. 8).

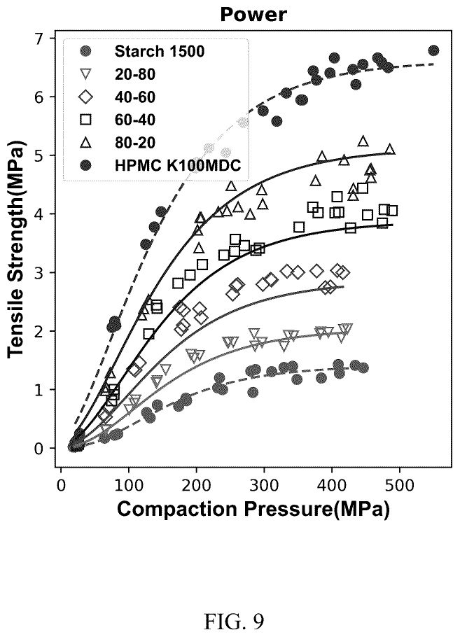

Starch is a well-known viscoelastic material that is commonly used in tablet formulations. Hence, it was used as a model viscoelastic material to prepare mixtures with the plastic excipient HPMC. The tabletability profiles (as determined according the explanations above) of the pure components (dashed lines) and mixtures for the HPMC K100MDC-Starch 1500 mixture system is shown in FIG. 9 and reflects agreement between the predicted tabletability profile and experimental data.

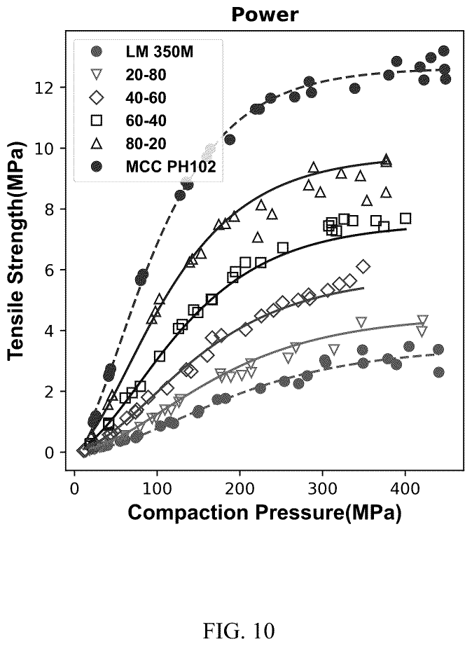

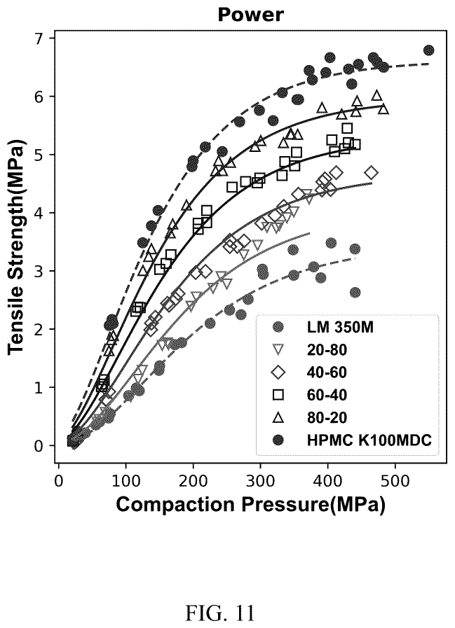

Plastic-brittle binary mixtures are routinely used in formulation to maintain a balanced mechanical property and tableting performance. Hence, commonly used plastic excipients, MCC and HPMC, and common brittle excipient Lactose were used to test the prediction model. The tabletability profiles (as determined according to the explanations above) of the pure components (dashed lines) and mixtures for the MCC PH102-LM 350M mixture system is shown in FIG. 10 and reflects agreement between the predicted tabletability profile and experimental data. The tabletability profiles (as determined according to the explanations above) of the pure components (dashed lines) and mixtures for the HPMC K100MDC-LM 350M mixture system is shown in FIG. 11 and reflects agreement between the predicted tabletability profile and experimental data.

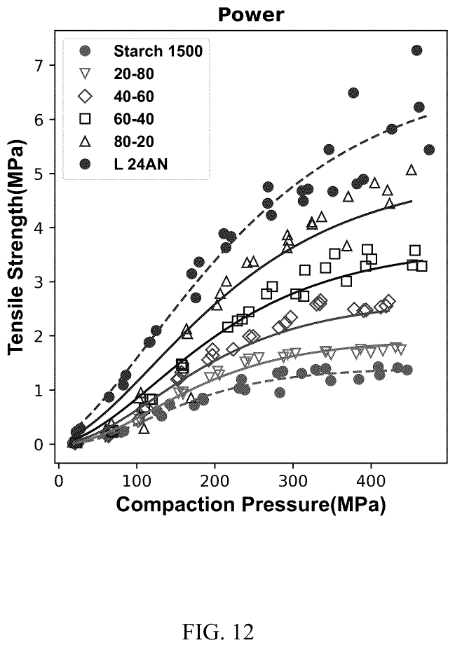

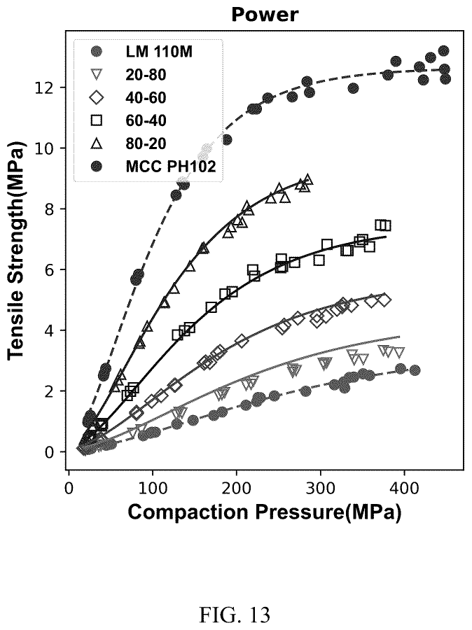

The brittle material Lactose 24AN used in the brittle-viscoelastic mixture system is relatively more compressible than those used in the Plastic-Brittle systems, i.e., LM 350M. The use of a set of different brittle excipient here is intended to broaden the range of properties of excipient. The tabletability profiles (as determined according the explanations above) of the pure components (dashed lines) and mixtures for the Lactose 24AN-Starch 1500 mixture system is shown in FIG. 12 and reflects agreement between the predicted tabletability profile and experimental data.

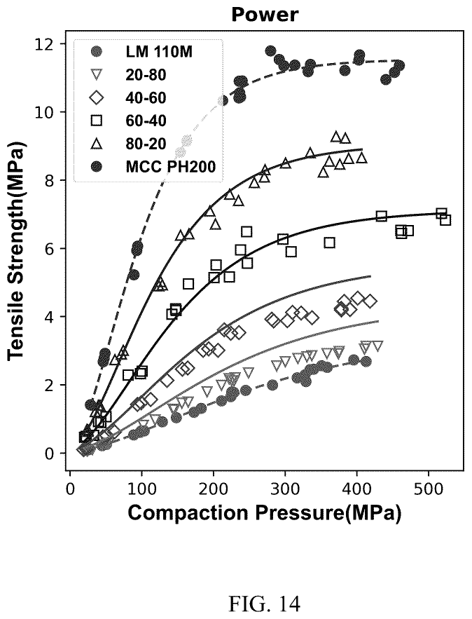

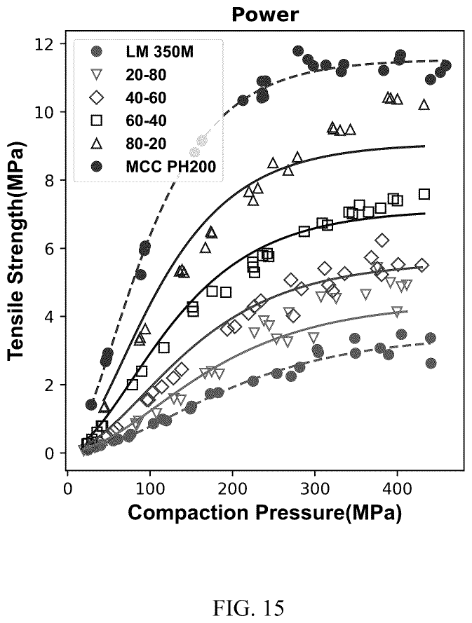

In addition to mechanical properties, particle size is also known to affect tabletability. Thus, experiments were performed to test the performance of models in the case that particle size of one of the components is changed. For this purpose, commonly used MCC-Lactose mixture system were considered. Two grades of MCC of different particle sizes, i.e., MCC PH102 (d₅₀=100 micrometer (μm)) and MCC PH200 (d₅₀=180 μm), were mixed with Lactose of two different particle sizes, LM 350M (d₅₀=35 μm) and LM 110M (d₅₀=130 μm). The tabletability profiles (as determined according to the explanations above) of the pure components (dashed lines) and mixtures for the MCC PH102-LM 110M mixture system is shown in FIG. 13 and reflects agreement between the predicted tabletability profile and experimental data. The tabletability profiles (as determined according to the explanations above) of the pure components (dashed lines) and mixtures for the MCC PH200-LM 110M mixture system is shown in FIG. 14 and reflects agreement between the predicted tabletability profile and experimental data. The tabletability profiles (as determined according the explanations above) of the pure components (dashed lines) and mixtures for the MCC PH200-LM 350M mixture system is shown in FIG. 15 and reflects agreement between the predicted tabletability profile and experimental data.

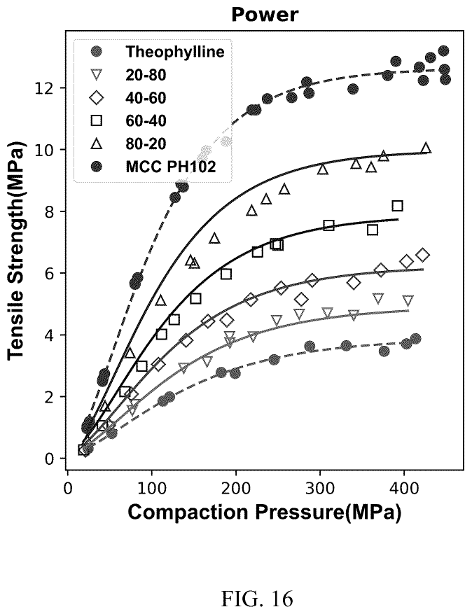

The results above from testing a large number of mixtures of excipients with diverse mechanical properties confirm the excellent performance of the systems and methods of the present disclosure in predicting tabletability of mixtures. Additional examples were prepared and reviewed to demonstrate applicability using an API. In particular, theophylline anhydrate (form II) was used as a model API that was mixed with MCC PH102. The tabletability profiles (as determined according to the explanations above) of the pure components (dashed lines) and mixtures for the MCC PH102-Theophylline mixture system is shown in FIG. 16 and reflects agreement between the predicted tabletability profile and experimental data.

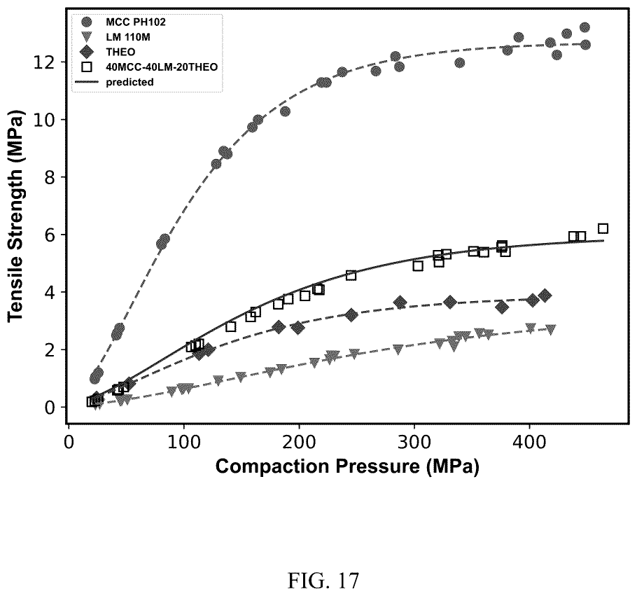

Additional examples were prepared and predicted tabletability profiles generated for a ternary mixing system of MCC PH102, LM 100M, and theophylline (“THEO”) (at a 40-40-20 mixing ratio). The tabletability profiles (as determined according to the explanations above) of the pure components (dashed lines) and mixtures for the 40MCC PH102-40LM-20THEO mixture system is shown in FIG. 17 and reflects agreement between the predicted tabletability profile and experimental data.

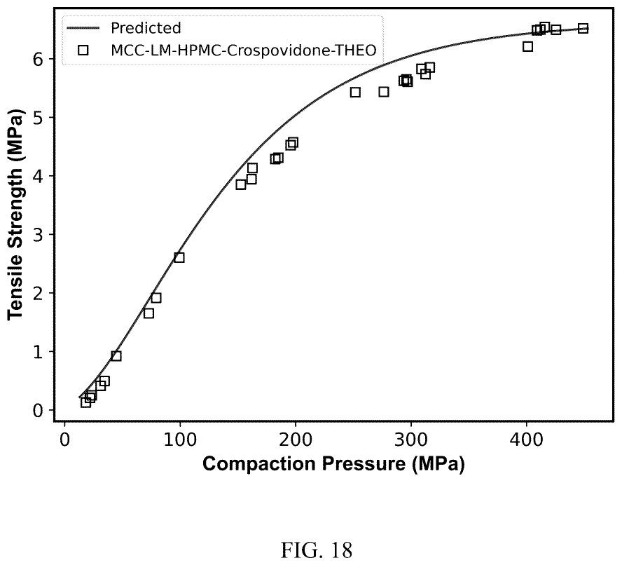

Additional examples were prepared and predicted tabletability profiles generated for a five component mixing system of MCC, LM, HPMC, Crospovidone, and theophylline (“THEO”). The tabletability profile (as determined according to the explanations above) for the MCC-LM-HPMC-Crospovidone-THEO mixture system along with corresponding experimental data is shown in FIG. 18 and reflects agreement between the predicted tabletability profile and experimental data.

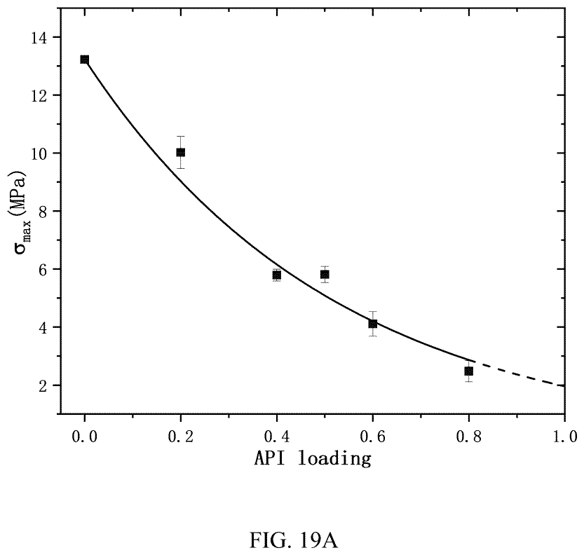

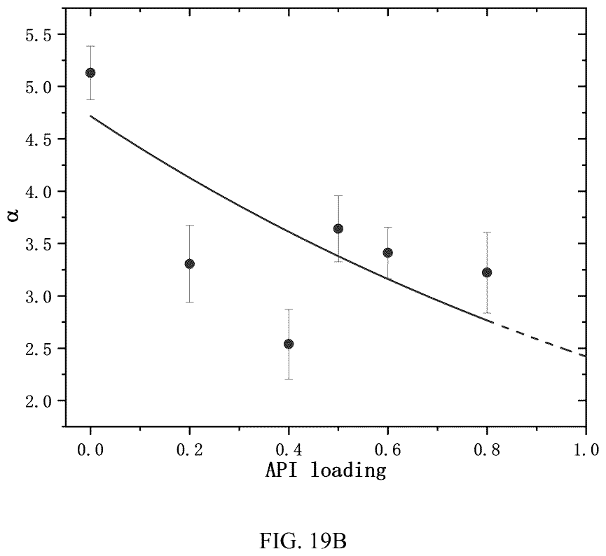

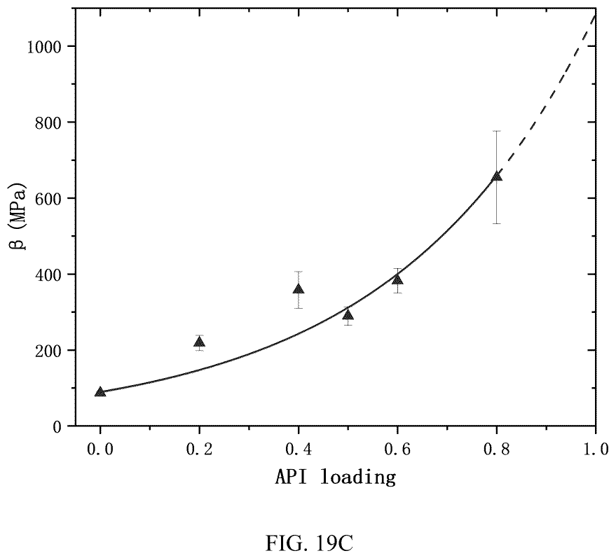

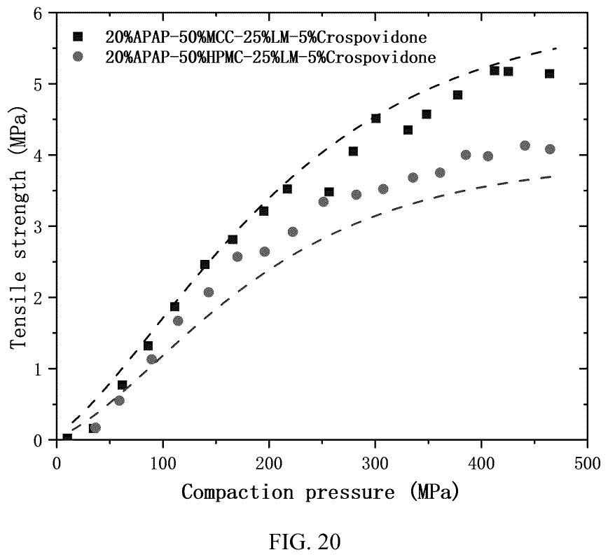

To evaluate an ability to obtain tabletability parameters of poorly compressible APIs in accordance with some methods of the present disclosure, acetaminophen and alanine were considered. In one example, binary mixtures of acetaminophen (“APAP”) and microcrystalline cellulose (“MCC”) were prepared at different APAP loadings, and tableted. Tabletability data of the APAP-MCC tablets were obtained and fitted to Equation 1 (the Vreeman-Sun equation) to obtain the three tabletability parameters αₘₐₓ, α, and β. The so-obtained tabletability parameters were plotted as a function of the APAP loading in the different mixtures as reported in FIGS. 19A-19C. Hypothetical tabletability parameters of APAP were determined by extrapolation; extrapolating the function to APAP ratio of 1 yielded the hypothetical parameter for pure APAP. FIG. 20 reports the predicted tabletability (lines) and experimental tabletability (points) for APAP and reflects strong agreement between predicted and experimental.

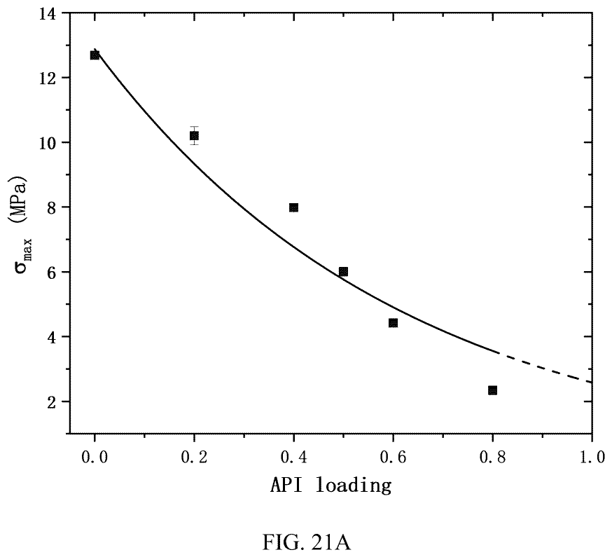

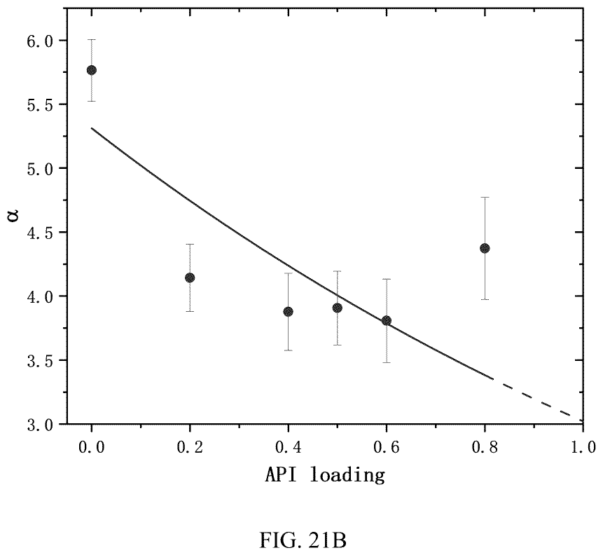

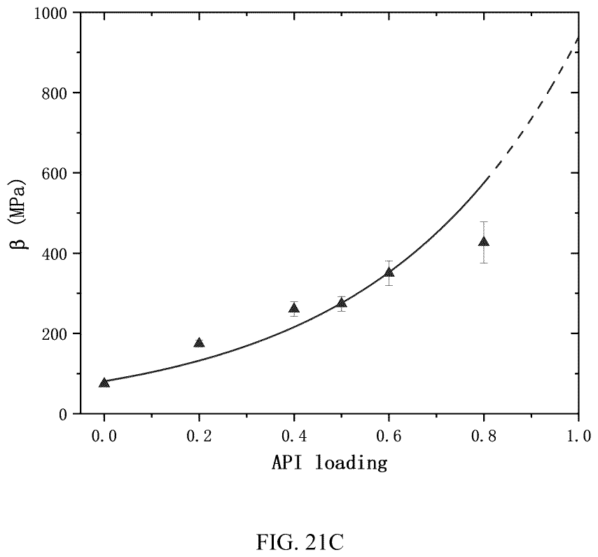

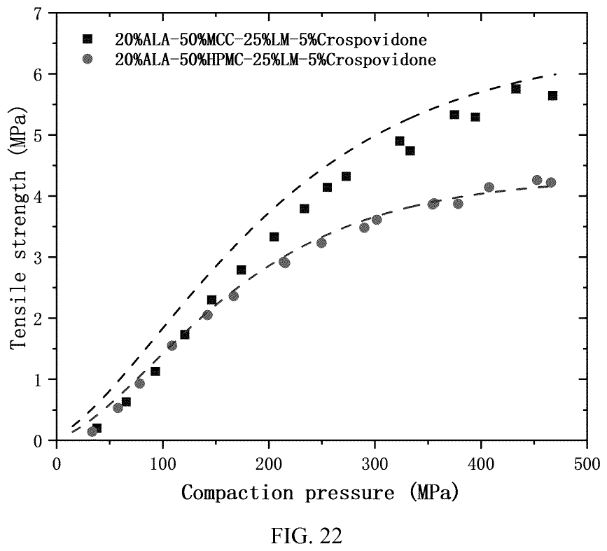

In another example, binary mixtures of alanine (“ALA”) and microcrystalline cellulose (“MCC”) were prepared at different ALA loadings, and tableted. Tabletability data of the ALA-MCC tablets were obtained and fitted to Equation 1 (the Vreeman-Sun equation) to obtain the three tabletability parameters σₘₐₓ, α, and β. The so-obtained tabletability parameters were plotted as a function of the ALA loading in the different mixtures as reported in FIGS. 21A-21C. Hypothetical tabletability parameters of ALA were determined by extrapolation; extrapolating the function to ALA ratio of 1 yielded the hypothetical parameter for pure ALA. FIG. 22 reports the predicted tabletability (lines) and experimental tabletability (points) for ALA and reflects strong agreement between predicted and experimental.

Examples were prepared to evaluate usefulness of systems and methods of the present disclosure to predict tabletability profiles of mixtures containing a lubricant. As a point of reference, the use of lubricant is common in tablet manufacturing and the incorporation of lubricant may cause deteriorated tabletability. Testing was performed to confirm that prediction systems and methods of the present disclosure are applicable to mixtures containing different levels of lubricants.

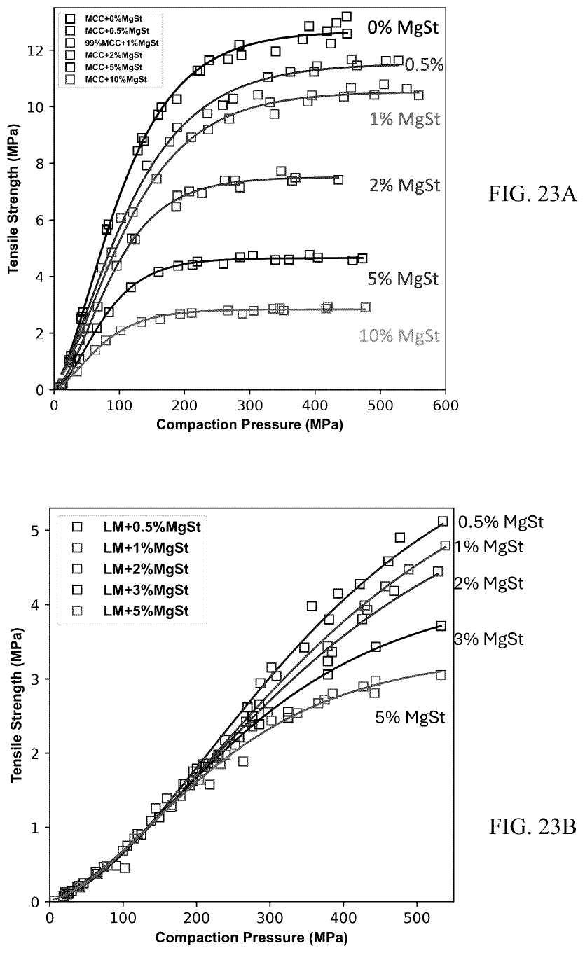

Use of magnesium stearate (MgSt) as a lubricant with a binary mixture of 60% MCC and 40% lactose monohydrate (LM) was evaluated at different loading levels, including 0.5% MgSt, 1% MgSt, 2% MgSt, and 5% MgSt. Sample mixtures of MCC and MgSt at MgSt loading levels of 0.5%, 1%, 2%, 5%, and 10% were prepared and tabletability determined as reported in FIG. 23A. Samples mixtures of LM and MgSt at MgSt loading levels of 0.5%, 1%, 2%, 3%, and 5% were prepared and tabletability determined as reported in FIG. 23B. The prediction methods or models of the present disclosure were then applied to predict tabletability of a lubricated mixture containing 60% MCC, 40% LM, and MgSt at MgSt loading levels of 0.5% MgSt, 1% MgSt, 2% MgSt, and 5% MgSt. For comparison, sample mixtures of 60% MCC, 40% LM, and MgSt at each of the MgSt levels under consideration (i.e., 0.5% MgSt, 1% MgSt, 2% MgSt, and 5% MgSt) were prepared, tableted and tabletability determined.

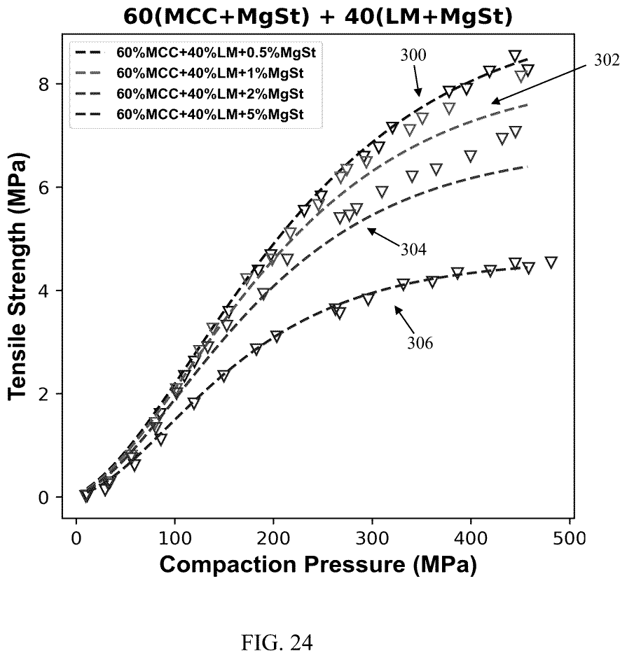

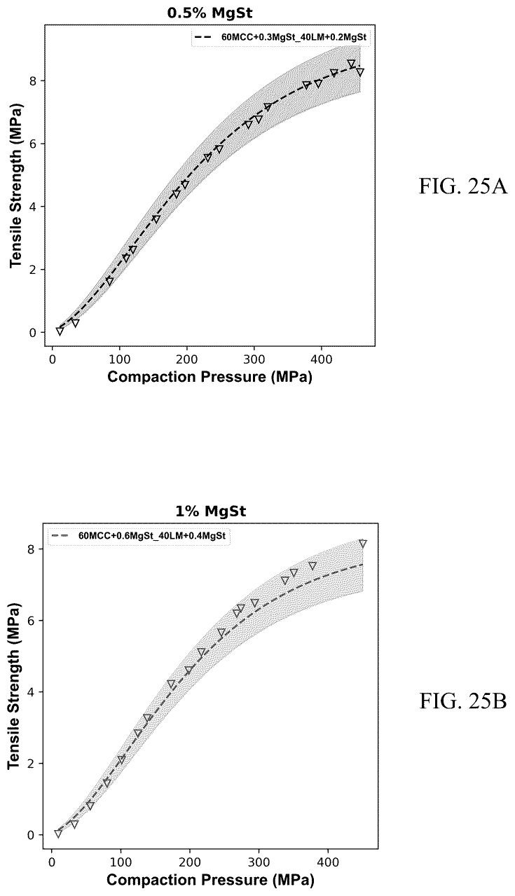

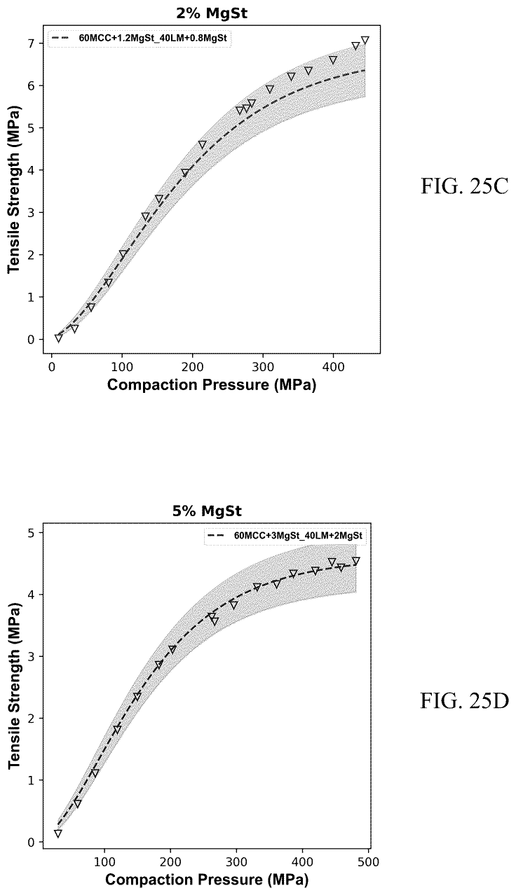

FIG. 24 reports the predicted tabletability plots (lines) and experimental plots (points) for each of 60% MCC+40% LM+0.5% MgSt (generally identified at 300), 60% MCC+40% LM+1% MgSt (generally identified at 302), 60% MCC+40% LM+2% MgSt (generally identified at 304), and 60% MCC+40% LM+5% MgSt (generally identified at 306). FIG. 25A provides the predicted tabletability plot (line) and experimental plot (points) for the 60% MCC+40% LM+0.5% MgSt examples and highlights that the experimental tabletability falls within 95% confidence interval of the predicted tabletability profile. FIG. 25B provides the predicted tabletability plot (line) and experimental plot (points) for the 60% MCC+40% LM+1% MgSt examples and highlights that the experimental tabletability falls within 95% confidence interval of the predicted tabletability profile. FIG. 25C provides the predicted tabletability plot (line) and experimental plot (points) for the 60% MCC+40% LM+2% MgSt examples and highlights that the experimental tabletability falls within 95% confidence interval of the predicted tabletability profile. FIG. 25D provides the predicted tabletability plot (line) and experimental plot (points) for the 60% MCC+40% LM+5% MgSt examples and highlights that the experimental tabletability falls within 95% confidence interval of the predicted tabletability profile.

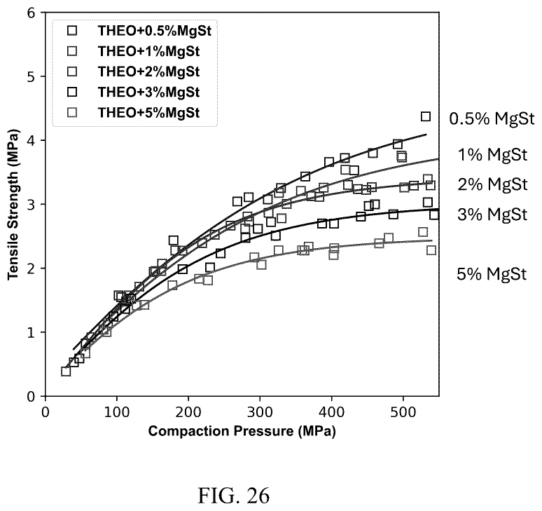

Additional experiments were performed to evaluate usefulness of systems and methods of the present disclosure to predict tabletability of lubricated mixtures of theophylline (“THEO”), MCC, and LM, in particular with MgSt as a lubricant at different loading levels. Tabletability of MCC and MgSt mixtures at MgSt loading levels of 0.5%, 1%, 2%, 5%, and 10% were determined as reported in FIG. 23A; tabletability of LM and MgSt mixtures at MgSt loading levels of 0.5%, 1%, 2%, 3%, and 5% were prepared and tabletability determined as reported in FIG. 23B. Samples mixtures of THEO and MgSt at MgSt loading levels of 0.5%, 1%, 2%, 3%, and 5% were prepared and tabletability determined as reported in FIG. 26. The prediction methods or models of the present disclosure where then applied to predict tabletability of the formulations of the Table below (in which all formulation composition components are expressed as %, w/w).

TABLE

| Ingredient | F1 | F2 | F3 | F4 |
|---|---|---|---|---|
| Theophylline | 25 | 35 | 50 | 50 |
| MCC 102 | 41.5 | 31.5 | 26.5 | 25 |
| LM 350M | 30 | 30 | 20 | 20 |
| Croscamellose sodium (CCS) | 3 | 3 | 3 | 3 |
| Magnesium stearate | 0.5 | 0.5 | 0.5 | 2 |
| Total | 100 | 100 | 100 | 100 |

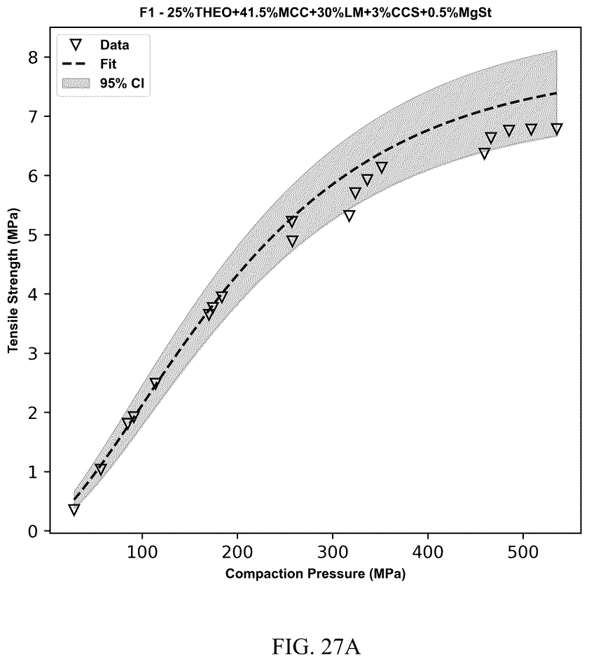

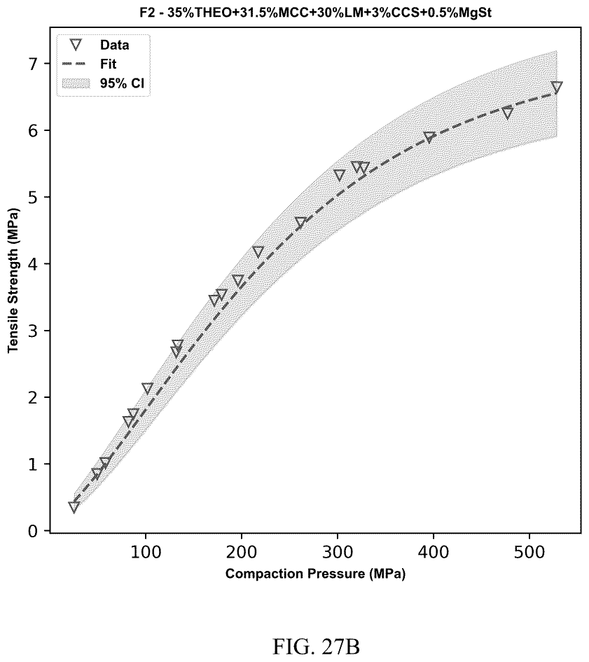

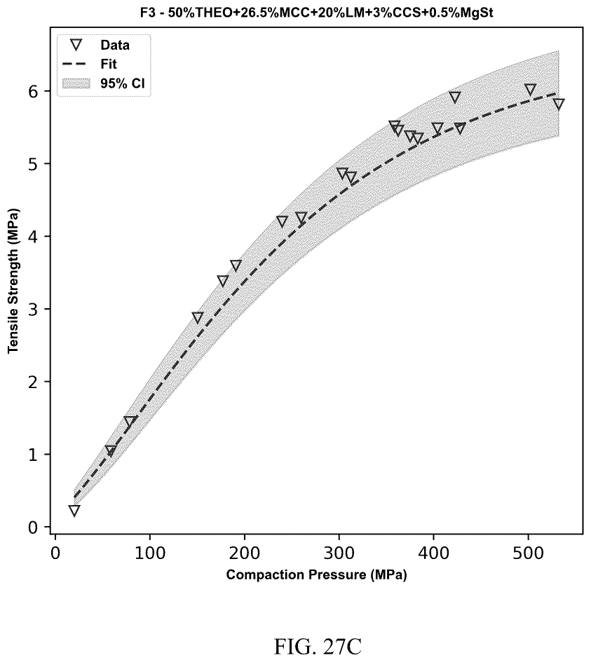

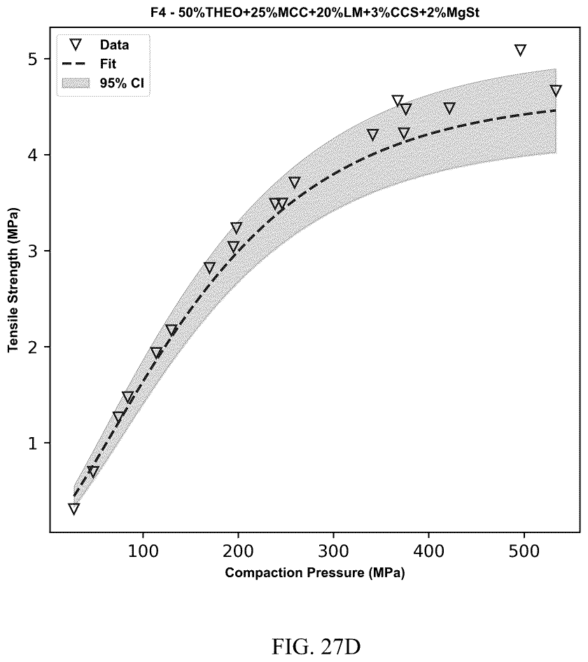

For comparison, sample mixtures of each of formulations F1, F2, F3, F4 as set forth in the Table above were prepared, tableted and tabletability determined. FIG. 27A provides the predicted tabletability plot (line) and experimental plot (points) for example formulation F1 (25% THEO+41.5% MCC+30% LM+3% CCS+0.5% MgSt) and highlights that the experimental tabletability falls within 95% confidence interval of the predicted tabletability profile. FIG. 27B provides the predicted tabletability plot (line) and experimental plot (points) for example formulation F2 (35% THEO+31.5% MCC+30% LM+3% CCS+0.5% MgSt) and highlights that the experimental tabletability falls within 95% confidence interval of the predicted tabletability profile. FIG. 27C provides the predicted tabletability plot (line) and experimental plot (points) for example formulation F3 (50% THEO+26.5% MCC+20% LM+3% CCS+0.5% MgSt) and highlights that the experimental tabletability falls within 95% confidence interval of the predicted tabletability profile. FIG. 27D provides the predicted tabletability plot (line) and experimental plot (points) for example formulation F4 (50% THEO+25% MCC+20% LM+3% CCS+2% MgSt) and highlights that the experimental tabletability falls within 95% confidence interval of the predicted tabletability profile.

The systems and methods of the present disclosure provide a marked improvement over previous designs. The predicted tabletability profiles in accordance with principles of the present disclosure were found to match well with the experimental tabletability for a plethora of binary mixtures, ternary mixtures, and a five-component mixture consisting of materials exhibiting a wide variety of mechanical properties. The surprisingly predictive approaches of the present disclosure represent a significant advancement that may enable material-sparing and expedited development of high-quality tablet products in the pharmaceutical industry. For example, an API mixture composition exhibiting acceptable tabletability could be predicted, within an acceptable range of accuracy, for any excipient of interest using systems and methods of the present disclosure without performing laboratory experiments. One can predict tabletability of a formulation once the tabletability of an active pharmaceutical ingredient (or its mixture with another excipient) and the tabletability of all mixture components is determined. This allows a formulator to screen tabletability of a potential formulation on a computer. Then, the lead formulation can be verified and optimized by running a few experiments. This technique is ground-breaking to the pharmaceutical industry.

Although the present disclosure has been described with reference to preferred embodiments, workers skilled in the art will recognize that changes can be made in form and detail without departing from the spirit and scope of the present disclosure.

What is claimed is:

1. A method comprising:
receiving from a formulator a proposed formulation comprising a plurality of components;
retrieving tabletability parameters for each component of the plurality of components;
generating a predicted tabletability profile for the proposed formulation based upon the tabletability parameters of each component of the plurality of components and a composition of the proposed formulation; and
conveying the predicted tabletability profile to the formulator.

2. The method of claim 1, wherein the tabletability parameters include at least one of:
a maximum strength attainable by the material upon complete densification (σₘₐₓ);
a sigmoidal profile shift influencing the onset of appreciable mechanical strength gain (α); and
a material plasticity (β).

3. The method of claim 2, wherein the tabletability parameters are determined by regression of experimental data to:
$$σ = {σ}_{m ax} {e}^{α {W}^{( - {e}^{\frac{- P}{β} - 1} )}} .$$

4. The method of claim 1, wherein the step of generating a predicted tabletability profile includes predicting tabletability parameters of the proposed formulation.

5. The method of claim 4, wherein the step of predicting tabletability parameters of the proposed formulation includes deriving a tabletability parameter of the proposed formulation based upon the corresponding tabletability parameter of each component of the plurality of components and a composition of each component of the plurality of components in the proposed formulation.

6. The method of claim 5, wherein the step of driving a tabletability parameter of the proposed formulation is based upon a mixing rule.

7. The method of claim 6, wherein the mixing rule is:
$${X}_{mix} = {\prod }_{i = 1}^{n} {X}_{i}^{{C}_{i}}$$
where Xᵢ is the tabletability parameters of a given mixture, and Cᵢ is composition of individual components.

8. The method of claim 4, wherein the step of generating a predicted tabletability profile further includes fitting the predicted tabletability parameters of the proposed formulation to a tabletability equation.

9. The method of claim 8, wherein the tabletability equation is:
$$σ = {σ}_{m ax} {e}^{α {W}^{( - {e}^{\frac{- P}{β} - 1} )}} .$$

10. The method of claim 1, wherein the tabletability parameters for a first component of the plurality of components is determined by:
obtaining tabletability data of a binary mixture comprised of the first component combined with a second component for at least two different first component loading levels; and
extrapolating tabletability parameters for the first component based upon the obtained tabletability data of the binary mixture.

11. The method of claim 1, wherein the tabletability parameters for a first component of the plurality of components comprises tabletability parameters of a mixture of the first component combined with a lubricant.

12. A system for predicting a tabletability profile of a proposed formulation comprising at least a first component and a second component, the system comprising:
an electronic device including an input device for receiving a proposed formulation from a user and a display device;
a prediction module; and
a material database;
wherein the prediction module is programmed to:
reference the material database and determine tabletability parameters for each of the components of the proposed formulation,
generate a predicted tabletability profile for the proposed formulation based upon the tabletability parameters of each of the components of the proposed formulation, and
prompt the display device to convey the predicted tabletability profile to the user.

13. The system of claim 12, wherein the tabletability parameters include at least one of:
a maximum strength attainable by the material upon complete densification (σₘₐₓ);
a sigmoidal profile shift influencing the onset of appreciable mechanical strength gain (α); and
a material plasticity (β).

14. The system of claim 12, wherein the material database includes tensile strength versus compaction pressure data for a first material, and further wherein the prediction module is programmed to determine tabletability parameters for the first material by regression of the tensile strength versus compaction pressure data as:
$$σ = {σ}_{m ax} {e}^{α {W}^{( - {e}^{\frac{- P}{β} - 1} )}} .$$

15. The system of claim 12, wherein the prediction module is programmed to generate a predicted tabletability profile by predicting tabletability parameters of the proposed formulation.

16. The system of claim 15, wherein the prediction module is programmed to predict tabletability parameters of the proposed formulation by deriving a tabletability parameter of the proposed formulation based upon the corresponding tabletability parameter of each component of the plurality of components and a composition of each component of the plurality of components in the proposed formulation.

17. The system of claim 12, the prediction module is programmed to generate a predicted tabletability profile by fitting the predicted tabletability parameters of the proposed formulation to a tabletability equation.

18. The system of claim 17, wherein the tabletability equation is:
$$σ = {σ}_{m ax} {e}^{α {W}^{( - {e}^{\frac{- P}{β} - 1} )}} .$$

19. The system of claim 12, wherein the material database includes tabletability properties of a mixture comprised of a component material combined with a lubricant.
